SEQUENCE LISTING (1 sequence)

SEQ ID NO: 1 - DNA, 1044 bp, Homo Sapiens
atggattatcaagtgtcaagtccaatctatgacatcaattattatacatcgagccctgccaaaaaatcaatgtgaagcaaatcgcagcccgcctcctgcctccgctctatcactggtgttcatctttggttttgtgggcaacatgctggtcatcctcatcctgataaatgcaaaaggctgaagagcatgactgacatctacctgctcaacctggccatctctgaccttttttccttcttactgtccccttctgggctcactatgctgccgcccagtgggactttggaatacaatgtgtcaactcttgacagggctctattttataggcttcttctctggaatcttcttcatcatcctcctgacaatcgataggtacctggctgtcgtccatgctgtgtttgctttaaagccaggacggtcacctttggggtggtgacaagtgtgatcacttgggtggtggctgttttgcgtctctcccaggaatcatctttaccagatctcaaaaagaaggtcttcattacactgcagctctcattttccatacagtcagtatcaattctggaagaatttccagacattaaagatagtcatcttggggctggtcctgccgctgcttgtcatggtcatctgctactcgggaatctaaaaactctgcttcggtgtcgaaatgagaagaagaggcacagggctggaggcttatttcaccatcatgattgtttattttctcttctgggctccctacaacattgtccttctcctgaacaccttccaggaattctttggcctgaataattgcagtagctctaacaggttggaccagctatgcaggtgacagagactcttgggatgacgcactgctgcatcaaccccatcatctagcctttgtcggggagaagttcagaaactacctcttagtcttcttccaaaagcacattgcaaacgcttctgcaaatgctgttctattttccagcaagaggctcccgagcgagcaagctcgtttacacccgatccactggggagcaggaaatatctgtgggcttgtga

(19) United States
(12) Patent Application Publication
(10) Pub. No.: US 2021/0163935 A1
(43) Pub. Date: Jun. 3, 2021

(54) Targeted Critical Fluid Nanoparticles Platform for Delivery of Nucleic Acids for Treatment of HIV-1 and Other Diseases

(71) Applicant: Trevor P. Castor, Arlington, MA (US)

(72) Inventor: Trevor P. Castor, Arlington, MA (US)

(73) Assignee: Trevor P. Castor, Woburn, MA (US)

(21) Appl. No.: 17/013,585

(22) Filed: Sep. 5, 2020

Related U.S. Application Data
(60) Provisional application No. 62/896,481, filed on Sep. 5, 2019.

(51) Int. Cl. C12N 15/113; C12N 9/22; A61K 47/10; C07K 14/52; B82Y 5/00

(52) U.S. Cl. CPC C12N 15/113; C12N 9/22; C12N 2310/20; C07K 14/521; B82Y 5/00; A61K 47/10

(57) ABSTRACT

Embodiments of the present invention are directed to an apparatus and methods for improved delivery of therapeutics and biologics for the treatment of diseases, such as HIV. Embodiments include the creation of nanoparticles for encapsulating nucleic acid. In some embodiments, the nucleic acid encapsulating nanoparticles are produced by a SuperFluids™ process, which results in particle size in the range of 100 nm to 200 nm. Further embodiments co-encapsulate nuclear acid with guide RNA molecules in the aqueous nanosomes core and targeting ligands on the surface of long circulating pegylated nanoparticles.

### RELATED APPLICATIONS

This application is related in part of U.S. Pat. No. 9,981,238 issued on May 29, 2018 which is incorporated by reference herein in its entirety.

### FIELD OF THE INVENTION

The present invention pertains therapeutic delivery methods and processes for nucleic acids (NAs) and other biologics in phospholipid nanoparticles for the improved delivery of CRISPR Cas9 and other biologics to targeted diseased human or animal cells and apparatus and methods for making the same.

### BACKGROUND OF THE INVENTION

There are at least 7,000 rare diseases from which between 25-30 million Americans are affected without a cure, and multiple chronic infectious diseases like chronic or latent HIV/AIDS and hepatitis C virus (HCV) from which >100 million global population is affected. The large number of diseases affecting millions of Americans and the global population have no long-term cures and are difficult to treat. These include rare monogenetic disorders/conditions, and chronic infectious and noncommunicable diseases such as: (i) Tay-Sach's syndrome; (ii) familial Alzheimer's disease type 4; (iii) chronic human immunodeficiency virus (HV) resulting from latent infection reservoirs that are inaccessible to antiretroviral drugs; and (iv) diabetes; and (v) cancer.

For these conditions without a current cure, nucleic acids (NAs) carry a huge potential for breakthrough therapeutic discovery. But in order to harness the therapeutic potential of NA, delivery platforms must be developed that are efficient and specific for the target tissues, yet broad enough to be applied across disease states using alternate drug content.

### SUMMARY OF THE INVENTION

There is increasing consensus that, for these hard-to-cure conditions, genetic signatures exist in human hosts that would inform novel therapeutic discovery and treatment designs. Nucleic acids (NAs) have been specifically used to target disease-associated genes, and these strategies continue to offer hope in gene-based therapies for multiple conditions for which current drugs are either less effective or an effective treatment is totally lacking.

One embodiment includes a targeted platform for the delivery of nucleic acids such as CRISPR Cas9 to delete a gene that is critical to HIV-1 pathogenesis towards a cure. In another aspect, this platform will have applicability to other highly unmet medical needs such as Alzheimer's disease, diabetes and cancer.

A further embodiment includes a proprietary SuperFluids™ (SFS) technology for the manufacture and delivery of small (100 nm to 200±50 nm), stable and widely distributed Critical Fluid Nanosomes (CFN) that can be used as carriers of multiple NA, protein and small molecule therapeutics as a single nanosomal therapeutic cocktail. This platform is also efficient for delivery of drugs that do not dissolve in aqueous solvents such as the blood or cross organ barriers such as blood-brain barriers, and thus have been limited in efficacy.

Embodiments include a delivery platform in which the CFN™ nanoparticles are be used to encapsulate and co-deliver the RNA-protein hybrid therapeutic called “clustered regularly interspaced short palindromic repeats (CRISPR),” pre-loaded on Cas9 protein (CRISPR/Cas9) or other Cas proteins to specifically target: (i) HIV co-receptor CCR5 (CRISPR/Cas9-CCR5); and (ii) HIV co-receptor CXCR4 (CRISPR/Cas9-CXCR4) or both. In one embodiment, both constructs are encapsulated in the aqueous core due to their hydrophilic characteristics, with the measurable immediate aim of achieving HIV cure either through the introduction of resistance mutations for elimination of susceptible host reservoirs. We refer to this nanoparticle cocktail as CNAP™ (CFN co-encapsulation of a combination nucleic acid (NA)-protein therapeutic that is PEGylated).

In an additional embodiment, the CNAP™ nanoparticle is coated with CCL5, aka RANTES protein which is a CC chemokine with a molecular weight of 9,900 that competes with HIV gp120 to bind CCR5, a co-receptor for HIV and suppresses infection by CCR5-tropic HIV. Coating CCL5 on nanosomes will be achieved by incorporating phosphatidylethanolamine into the lipid bilayer during synthesis of the nanosomes. The ethanolamine on the surface of the nanosomes will then be cross-linked to the lysine residues in the RANTES protein by glutaraldehyde or other amine cross-linking chemistries. CCL5 is a natural ligand produced in the human body and CCR5 is one of its receptors. CCR5 serves as a co-receptor on cells that also express CD4 and together, the two molecules make the cells permissible to HIV infection. Hence, specific delivery to CD4+ cells expressing CCR5 protein will be provided by the presence of CCL5 on the surface of the nanosomes which would protect such cells from HV infection by the abolition of expression of CCR5 by the CRISPR/Cas9 hybrid.

In an additional embodiment, the CNAP™ nanoparticle is coated with an alternative natural ligand for CCR5. These include MIP-la (aka CCL3), MIP-10 (aka CCL4) and MCP-2 (aka CCL8). Of these, CCL4 is particularly advantageous since it's only known receptor is CCR5, which makes it highly specific for only the cells expressing CCR5. This specificity is not provided by the other natural ligands of CCR5 mentioned here since they can bind to one or few other receptors in addition to CCR5.

In additional embodiment, the CNAP™ nanoparticle is coated with a small molecule inhibitor of HIV binding to CCR5 such as Maraviroc or with truncated or altered CCL5, which compete with HIV binding to CCR5 with a higher efficiency than CCL5. Since Maraviroc is a hydrophobic compound poorly soluble in water, coating of the CNAP™ nanoparticle will be performed by simple mixing which will allow the compound to bind to the CNAP™ by hydrophobic interactions. The truncated or altered CCL5 proteins will be coated on to the CNAP™ by the same methodology as the one described above for CCL5.

In an additional embodiment, the CNAP™ nanoparticle is coated with SDF-1, aka CXCL12 protein which is a 72 amino acid long CXC chemokine with a molecular weight of 8,522 that competes with HIV gp120 to bind CXCR4, a co-receptor for HIV and suppresses HIV infection. CXCL12 is a natural ligand produced in the human body and CXCR4 is one of its receptors. CXCR4 serves as a co-receptor on cells that also express CD4 and together, the two molecules make the cells permissible to infection by CXCR4-tropic HIV. Hence, specific delivery to CD4+ cells expressing CXCR4 protein will be provided by the presence of CXCL12 on the surface of the nanosomes which would protect such cells from HIV infection by the abolition of expression of CXCR4 by the CRISPR/Cas9 hybrid.

In an additional embodiment, CNAP™ nanoparticle is coated with an alternate ligand for CXCR4. These include the different isoforms of CXCL12, 7 of which have been identified so far, and Plerixafor, a small molecule inhibitor of CXCL12 binding to CXCR4. The methodology for coating these alternative ligands will be the same as the ones described above for CCL5. The methodology described for coating the proteins on the surface of CNAP™ can also be used to coat Plerixafor since Plerixafor has a number of amine groups which can be used for cross linking with the ethanolamine head groups present on the surface of CNAP™.

In another aspect, this therapeutic is tested in cell culture models in vitro and in mice models of HIV latency in vivo to validate delivery efficiency, target specificity, tissue distribution, drug stability and efficacy as demonstrated by elimination of HIV in the animal after the stoppage of ART. CRISPR/Cas9-CCR5 or CRISPR/Cas9-CXCR4 when delivered efficiently, specifically and broadly, will introduce specific non-deleterious mutations functionally similar to CCR5delta32 mutations that will allow the development of a reservoir of CD4 cells resistant to HIV infection.

In one embodiment, a method is disclosed for constructing, formulating, manufacturing and characterizing CCR-5 specific long-lived nanosomes (CNAP™) containing genome editing CRISPR/Cas9-CCR5 coated with CCL5, the natural ligand for CCR5; and further validating CNAP™ delivery in vitro and in vivo in relevant disease use models. CRISPR is a guide RNA that leads the Cas9 protein to specific cell or viral targets for editing of the cognate genomic regions. In another aspect, the CNAP™ is coated polyethylene glycol (PEG) to increase residence or circulation time of the therapeutic in the body.

In some embodiments, a versatile therapeutic platform is disclosed that can be used to substitute for any disease target. A specific HIV disease model is used to demonstrate the versatility of the platform by targeting the host genome, while also addressing a global pandemic dilemma in the form of incurable HIV resulting from persistent cellular reservoirs of viral infection.

In another embodiment, a method is disclosed for evaluating the targeting of CNAP™ in vivo in preclinical animal models, including effectiveness, toxicity, efficacy, and mechanism of action studies; as well as methods for the scale-up manufacturing of nucleic acid-based combination therapeutics.

### BRIEF DESCRIPTION OF THE DRAWINGS

FIG. 1 depicts CNAP™ nanoparticles (aka nanosomes), embodying features of the present invention;

FIG. 2 illustrates gRNA sequences to target CCR5 expression, showing the 32-bp deletion;

FIG. 3 shows a schematic illustration of an apparatus for making said CNAP™ nanoparticles of present invention; and

FIG. 4 depicts a more detailed illustration of an apparatus for making CNAP™ nanoparticles according the present invention.

### DETAILED DESCRIPTION OF THE INVENTION

Nucleic acid (NA) based Therapeutics: Nucleic acids (NAs) are novel potential therapeutics for multiple disease targets, and are thus a versatile tool for the treatment of several conditions for which current drugs are either less effective or an effective treatment is totally lacking. These include potential treatments for the over 7,000 rare diseases from which between 25-30 million Americans are affected without a cure, and multiple chronic infectious diseases like HIV/AIDS and hepatitis C virus (HCV) affecting >100 million global population. There is an increasing consensus that, for these hard-to-cure conditions, genetic signatures exist in human hosts that would inform novel treatment designs. The biggest impediment however, to harnessing the therapeutic value and breadth on NAs for gene-based therapeutics is the lack of optimal delivery platform to efficiently and in a stable long-term efficacious manner, introduce these products to specific disease targets.

Nanotechnology can serve as a vehicle-free platform for both broad and specific NA delivery. Limited but notable progress has been made towards the delivery of certain types of NAs into some tissues, including delivery to the liver and bone marrow derived stem cells. But there still remains a global need for novel delivery vehicles that are specific enough to target different tissues and cell types, yet broad enough to be useful across various disease states. Due to their larger size and limited stability relative to small molecule drugs, effective delivery of NAs has often relied on viral carrier vectors such as lentivirus, adenovirus and AAV that suffer from drawbacks such as safety and lack of efficacy due to neutralization by host antibodies. Moreover, emerging gene-based therapeutics increasingly require targeted co-delivery incorporating both a ‘guide’ NA and proteins to maximize functional effects. Such strategies include the use of: (i) zinc finger nucleases (ZFN) to edit the HIV coreceptor gene CCR5 for purposes of establishing a cure against HIV, and (ii) CRISPR-(clustered regularly interspaced short palindromic repeats)mediated gene editing for broader therapeutic application to genetic and infectious disease conditions including HIV, Alzheimer's disease, sickle cell and beta-thalassemia's. For these gene-based therapies to succeed, compatible and effective delivery platforms must be readily available to researchers testing candidate products and clinicians delivering care. A lipid-based nanoparticle technology has been used for decades to introduce therapeutic agents including NAs to tissue targets, but this too, is yet to be successfully harnessed for multi-product therapeutics delivery.

SuperFluids™ (SFS) Technology. SuperFluids™ (SFS) technology can be used to manufacture and deliver highly pure Critical Fluid Nanosomes (CFN™) in the particle size range of 100 nm to 200 nm. CFN™ uses green technology that harnesses widely available environment friendly gases, exploiting the inherent thermodynamic properties of SuperFluids™ in a purely physical process that preserve purity, integrity and efficiency of nanosomal therapeutics. SuperFluids™ are supercritical, near-critical or critical fluids with or without polar cosolvents. The CFN™ process can be used for the efficient encapsulation siRNA and for co-encapsulation of both hydrophilic and hydrophobic molecules. The CFN™ platform can also be used to co-encapsulate combinations of NAs and small molecule therapeutics, making products relevant to latent HIV activation and cure and which are broadly applicable to gene delivery across disease boundaries. Specifically, the CFN™ process is used to formulate and manufacture therapeutics containing a CCR5specific CRISPR/Cas9, coated with a CCR5 receptor targeting molecule or a CXCR4 specific CRISPR/Cas9 coated with a CXCR4 receptor targeting molecule.

The choice of “disease use model” is strategic and deliberate: (i) HIV is an incurable infection that maintains a permanent latent state of infection in viral sanctuaries in the presence of combination antiretroviral therapy. Thus the HIV model offers a unique prototype for multiple tissue specificity and efficacy testing of the CFN™ delivery platform; (ii) there is a well-established genetic signature in certain human populations, the CCR5 delta32 gene mutation that is associated with absolute resistance to HIV infection, and which is at the center of HIV cure research; and (iii) naturally occurring ligands such as CCL5 which will specifically bind to immune cells carrying the CCR5 receptor on their cell surfaces.

One embodiment of the present invention is a method of CFN™ co-encapsulation of a combination nucleic acid (NA)-protein therapeutic that is PEGylated (CNAP™), and targeted by cell and genome-specific RNA molecules in high-circulation, widely distributed, small nanosomes for the delivery of complex drug formulations. This CNAP™ platform includes in one aspect the NA-protein hybrids CCR5-CRISPR/Cas9 in the aqueous core, and CCL5, the ligand for CCR5 receptor, on the surface of the nanoparticles. The Cas9 protein has a nuclear localization signal as a part of the protein sequence to allow nuclear localization of the NA-protein complex. Polyethylene glycol coating (PEGylation) is introduced during co-encapsulation and manufacturing to increase CNAP biological residence time and overall therapeutic efficacy therapeutic index. PEGylation may be customized to meet requirements for tissue contact time to alleviate toxicity and improve bioavailability. The CNAP is specifically designed to deliver the NA-protein complex to the nuclei of CCR5 expressing cells and delete the CCR5 gene of the target cells. This CNAP approach is capable of co-delivering therapeutic cocktails to fast track testing of treatment strategy for complex diseases for which cure is not available and single products are not effective.

In another aspect of the present invention, the cells expressing CCR5 can also be targeted by coating the CNAP with alternative ligands that bind to CCR5. These include protein ligands such as CCL3, CCL4, CCL8 and truncated or modified CCL5; and small molecule ligands such as Maraviroc.

In another aspect of the present invention, the cells expressing CXCR4, that can potentially support HIV infection, can be targeted by coating the CNAP with SDF-1 aka CXCL12, or its various isoforms or its small molecule analog, Plerixafor.

In another aspect of the present invention, the CNAP vehicle is broadly applicable; therapeutic content can be readily substituted with a wide array of drugs, both soluble and insoluble in aqueous environments such as blood. In a further aspect the CNAP will be coated with PDL-1 antibodies and T-cell surface markers (e.g., CD44 and CD54 antibodies) to enhance targeting to resting CD4 T-cells in latent reservoirs. Further embodiments include optional co-encapsulation with latency activating drugs to endogenously activate latent viral reservoirs.

The inventive strategy achieves three important objectives: (i) delivery of multiple compounds of different structures, including a nucleic acid-protein hybrid carrying RNA targeting signal, in the aqueous core of the nanoparticles vehicle; (ii) making a platform for broader application in complex disease use model; and (iii) targeting to specific cells. The scope of the therapeutic vehicle is not limited by therapeutic strategy; recently described tools that feature a hybrid between CRISPR/Cas9 and ZFN technology, using transposons to achieve gene modification (i.e. the TALENS), can be readily adapted in the CNAP vehicle for more efficient and targeted delivery. These CNAPs have a high therapeutic efficacy and the cocktail can be formulated for a wide range of administrative routes including intravenous, intramuscular, and intranasal, thus offering breadth, specificity and durability (bioavailability) in tissue and disease targets.

SuperFluids™ (SFS) and Critical Fluid Nanosomes (CFN). SuperFluids™ (SFS)—Critical Fluid Nanosomes (CFN™) are natural carriers of nucleic acid (NA) therapeutics and are efficient delivery platforms for multiple cellular targets including HIV latency as a “disease use model.” CFN™ technology has been tested for the encapsulation of a wide range of both hydrophobic and hydrophilic small molecules including nucleic acid therapies (siRNA). The CFN™ platform is efficient for co-encapsulating multiple therapeutic candidates in a nanosomes cocktail formulated with NA constructs targeting specific human and viral genes in cellular reservoirs and viral sanctuaries.

SFS-CFN™ can manufacture high quality and grade nanosomal therapeutic cocktail (CNAP) that demonstrate stability, breadth, delivery efficiency and potency in appropriate in vitro and in vivo experimental platforms for: (i) establishing the right design and formulation for stable, viable and long-lived nanosomal cocktails; (ii) further optimization of physical and chemical characteristics, including additional modifications that improve stability and functionality; and (iii) validation of effectiveness and preservation of integrity of the CNAP cocktail in relevant ‘disease use model.’

The HIV model is perfect for complex disease situations; while gene-based therapy has successfully worked in just two patients (the ‘Berlin’ patient and the ‘London’ patient) following bone marrow transplant of cells harboring gene mutants conferring resistance to HIV, reversal of ‘supposed cure’ in a few other patients (‘Boston’ patients and ‘Mississippi baby’), present important challenges to unimodal therapeutic designs. For such challenges, therapeutic efficacy can be improved using approaches that deliver combination therapeutics in a single cocktail to sustain a cure. Hence, our approach is to co-encapsulate CCR5-specific CRISPR/Cas9 nucleic acid-protein hybrids, with a small protein like the CCR5 ligand, CCL5, coated on the surface, in a nanosomal vehicle that are stabilized for tissue residence and durability through PEGylation. The CRISPR/Cas9 hybrids will be co-encapsulated in the aqueous core of the nanoparticles. The RNA-guided CRISPR/Cas9 is specific for target cells to facilitate gene editing, while CCL5 will target CCR5 expressing cells specifically. Moreover, a window exists to markers of resting state CD4 T-cells (such as using CD44 and CD54 antibodies) as ‘nanosomes guide’ to latently infected cellular reservoirs in additional platform validations.

Multiple Disease Application: The CFN™ platform strategy is readily applicable to multiple disease phenotypes; the CNAP cocktail can be designed to contain any range and type of therapeutics including nucleic acid products such as siRNA, mRNA, DNA or DNA vectors; small molecules; and proteins of varying polarities. Moreover, the CNAP vehicle can be targeted to any tissue type in vivo using specific guide molecules targeting genes or cell surface markers. Moreover, the CNAP cocktail can be delivered in vivo via a variety of administration routes to include intranasal sprays for hastened breach of blood-brain barrier as required for diseases such as Alzheimer's disease; intraperitoneal injections in Proof-of-Concept animal studies; intravenous administration for wide circulation of drugs hitherto insoluble in the aqueous blood; intramuscular injection for NA uptake and expression; and localized direct-to-organ/tissue application as would be necessary for diseased cancer tissues.

One goal of the present development is to construct, formulate, manufacture and characterize long-lived nanoparticles containing genome editing (a) CRISPR/Cas9-CCR5 and (b) coating them with CCL5, the ligand for CCR5 receptor. Delivery of several drugs is impeded by their limited solubility in the aqueous blood and extended reach to hidden tissues across membrane barriers. Nanosomes alleviate this weakness by ‘solubilizing’ both polar and non-polar drugs in their hydrophilic and hydrophobic compartments.

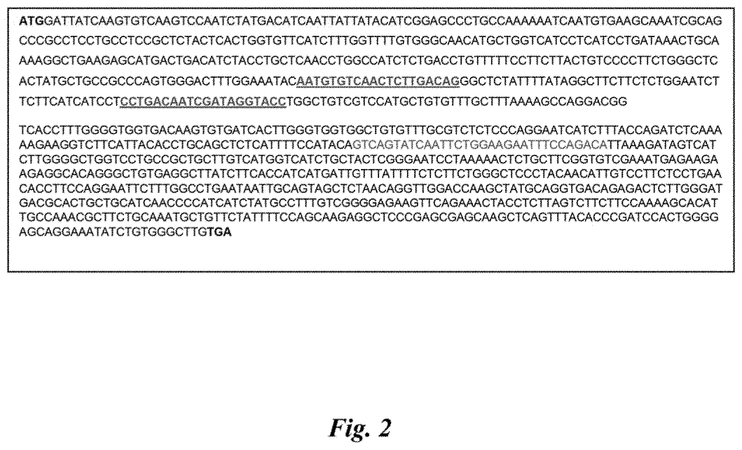

Design, Selection and Validation of CRISPR Nucleic Acid Targets: CRISPR/Cas9 is a special class of RNA-protein hybrids engineered to facilitate targeted gene editing. Each CRISPR/Cas9 complex consists of two functional domains: (i) a 20 nucleotide-long NA component called the guide RNA (gRNA) that recognizes target genomes interspaced by the trinucleotide 5′-NGG-3′ protospacer adjacent motif (PAM); and (ii) a protein (nuclease) domain comprised of the bacterial Cas9 system. The design and genome of CRISPR/Cas9 complex has been previously described. For CCR5, gRNA targeting sequence is designed to have the least similarity with the highly homologous CCR2 around the mutation region in order to minimize off-target effect. Additionally, a BLAST search was done on the NCBI database to eliminate candidate gRNA with unwanted off-target effects. Two such sequences, of several identified sequences, are shown in FIG. 2. These sequences are up stream of the delta-32 mutation in the ‘Berlin’ patient and would result in a protein which is even more truncated in that mutation. In addition, gRNA sequences are designed that will result in the 32-base pair deletion of the delta-32 mutation. The encapsulated Cas9 protein will carry an NLS, to allow transport to the nucleus. The designed complexes can be custom procured as RNA molecules from a commercial third party such as IDT or GenScript. Several CCR5 specific sequences are also commercially available from GenScript. Cas9 with NLS can be obtained from commercial sources such as GenScript. When the Cas9-NLS is co-encapsulated with the gRNA sequences, they form a complex which will be delivered to the cytoplasm of the CCR5 expressing cells by CCL5 present on the surface of the nanoparticles. Once inside the cytoplasm, the complex is directed to the nucleus by the NLS. When the complex is inside the nucleus, the gRNA sequences guide the Cas9 protein to the DNA sequence of the host genome targeted for deletion. Therefore, the different components of the nanosomes guide each other to the final site of action.

Reagents are validated in the laboratory for target specificity, efficiency and activity using GeneArt™ Genomic Cleavage Detection Kit (ThermoFisher or similar sources), a protocol that reliably and rapidly detects locus-specific double-strand break formation using primer-specific PCR and gel analyses. If necessitated by inconclusive results, western blotting is performed to confirm the gene truncation/deletion. If the use of single gRNA sequences does not result in efficient knock-down of CCR5 expression in the target cells, use two or more gRNA sequences along with a guide ssDNA sequence are used to ensure the required frameshift that would knock down the protein expression.

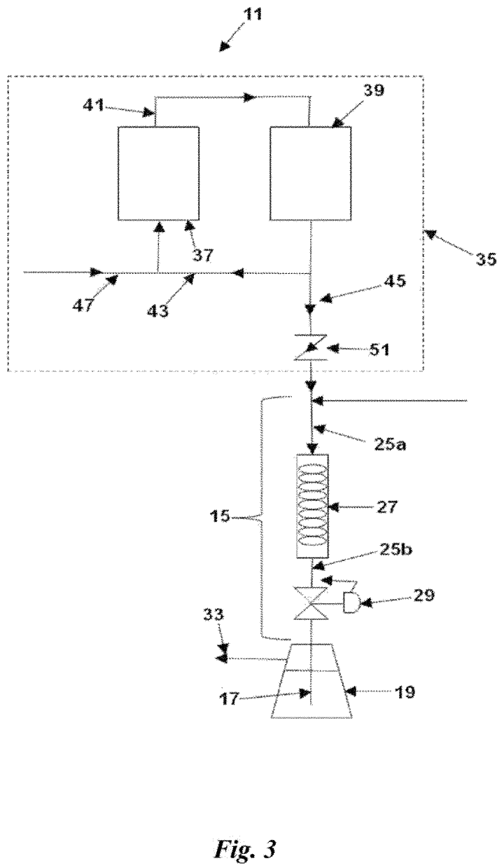

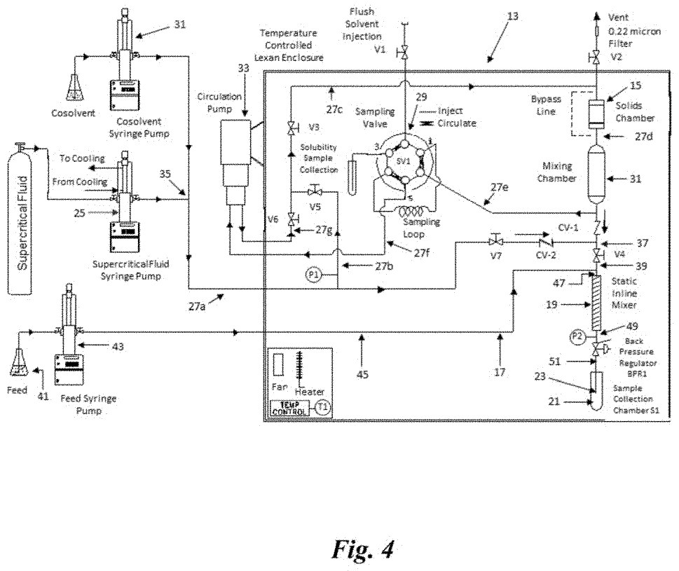

CNAP Construction and Formulation in SuperFluids™ (SFS) Solvents: The CFN™ process (shown in FIGS. 3 and 4) is used for the formation of small, uniform CNAP liposomes. This technology has produced nanosomes ranging in size from 100 nm to 200 nm, which are robustly capable of encapsulating multiple therapeutic components of different polar characteristics that include a nucleic acid, antibodies and proteins/enzymes for multipronged approach to gene-based therapy. In the CFN™ process, SFS with or without polar co-solvents at appropriate conditions of pressure and temperature are utilized to solvate phospholipids, cholesterol and other nanosomal raw materials. After a specific residence time, the resulting mixture is decompressed via a backpressure regulator (valve), with bubbles forming at the injection nozzle and nanosomes forming in the receiving buffer.

SuperFluids™: The selection of SuperFluids™ will depend on the solubility of the bilayer forming lipids and the hydrophobic active ingredient, Maraviroc. Based on our experience with similar molecules, we plan to use SFS propane and 20% ethanol at 3,000 psig and 40° C. unless suggested otherwise by thermodynamic, solubility and stability studies.

Phospholipid Raw Materials: Lipid materials will be selected on the basis of previous studies and the solubility of these lipids in the SFS under appropriate operational conditions. The lipid raw materials will consist of synthetic and derivatized phospholipids, including phosphatidylcholine (PC), phosphatidylglycerol (PG), phosphatidylserine (PS), dimyristoyl-phosphatidylcholine (DMPC), dimyristoylphosphatidylglycerol (DMPG), phosphatidyl-ethanolamine (PE) and polyethylene glycol conjugated distearylphosphatidylethanolamine (either DSPE-PEG₂₀₀₀ or DSPE-PEG₃₅₀₀) to provide higher serum/plasma retention. The molar ratio of total lipid to drug will be set ˜20:1. Nanosomal compositions that will be evaluated are listed in Table 1. The formulations listed in Table 1 are also found to be effective in the encapsulation of siRNA and similar hydrophobic molecules.

TABLE 1
Lipid Compositions and Molar Ratios

| PC:CH:PE | 1:1:1 and 2:1:1 |
|---|---|
| PC:PG:CH:PE | 1:0.1:0.4:0.4 |
| PC:PS:CH:PE | 1:0.1:0.4:0.4 |
| DMPC:DMPG:CH:PE | 1:0.1:0.4:0.4 |
| DMPC:DMPG:CH:PE:DSPE-PEG₂₀₀₀ | 1:0.1:0.4:0.35:0.05 |

Nanoencapsulation Procedure: The NA construct and Cas9-NLS are encapsulated in phospholipid nanosomes in the modified SFS-CFN apparatus shown in FIG. 2. The solids chamber is first loaded with liposomal raw materials which are then solubilized in the SFS-cosolvent mixture in the high-pressure circulation (HPC) loop by recirculation for 30 minutes. This lipid-enriched SFS stream is then mixed in a co-injection mode with a feed consisting of NA-Cas9 complex solution in an in-line static mixer, at the operating pressure, located downstream of the HPC loop but upstream of the back-pressure regulator. The resulting combined mixture is then decompressed into a biocompatible collection buffer. Due to the reduction in pressure, the solvated phospholipids deposit out at the phase boundary of the aqueous bubble. As the bubbles detach from the nozzle into the aqueous solution, they rupture, causing bilayers of phospholipids to peel off, thereby encapsulating solute molecules and spontaneously sealing themselves to form phospholipid nanosomes. Product volatilization and oxidation as well as processing time and organic solvent usage are significantly reduced with the use of SFS, thus greatly improving end-product purity and integrity. The PEGylated cocktail is encapsulated in the SFS phospholipid nanosomes apparatus shown in FIG. 2. The procedure is performed to provide a continuous flow rate of the components and allow a steady production of the product. Different nanosomes are produced by various lipid materials in the range of 100 to 200 (i 50) nm. Nanosomal suspensions can be filtered by a 0.22 μm filter as a final sterilization process.

CNAP Encapsulation and PEGylation: Molecules such as DNA or RNA are polyanionic, with net negative charges contributed by the 3′ phosphate groups that make them hydrophilic. Most proteins are oligomeric or polymeric with net positive charge. CRISPR/Cas9 complex has a net negative charge deriving from the RNA component. Thus, the polar (hydrophilic) complex is encapsulated in the aqueous core of the nanoparticles. SuperFluids™ are utilized to first solvate phospholipids as described above. The phospholipids are then be mixed with a solution of CCR5-specific CRISPR/NLS-Cas9 complex prior to decompression and injection into a biocompatible solution. This CFN™ technology has been used to form stable phospholipid nanosomes containing siRNA, Brefeldin A (BFA), Camptothecin (Top1) and Neomycin (Tdp1) as well as irinotecan (Top1) and tetracycline (Tdp1). To avoid phagocytosis of the nanosomes and dampening of their efficacy, the nanoparticles are coated with polyethylene glycol (PEG), a step that dramatically increases nanosomes residence time biodistribution. Commercially available phospholipids with head groups linked to PEG of various molecular weights are utilized. Low concentrations of PE are also included in the phospholipid mix to allow the presence of amine groups on the particle surfaces which would enable cross linking to the amine groups of CCL5. Additionally, another targeting step is used in which the nanosomes are coated with additional antibodies or other relevant cell surface markers to either guide the nanosomes to latently infected cells or stimulate immune cells such as CD8 and NK cells for enhanced nonspecific immunity as part of comprehensive in vivo platform. Thus, the CNAP provides room for targeting at two levels: (i) NA-guided targeting at genetic level, and (ii) particle targeting at cellular level.

Coating Nanoparticles with CCL5: CCL5 (aka RANTES protein) is a 9.9 KD protein which is a relatively small protein. The nanosomes will be prepared using PE as already described above. The amine groups on this protein such as the N-terminus and those carried by basic amino acids such as lysine are cross linked to the amine head group on the PE using glutaraldehyde or by using more specific amine cross linking chemistries.

Physical and Chemical Characterization: Smaller and more tightly packed nanosomes show longer circulation times in biological environments. Several operational conditions have been identified including temperature, nozzle size and rate of decompression that strongly influence nanosomes size distribution. Other parameters include phospholipid type (synthetic PEGylated or unpegylated), nanosomes composition and load, NA-Protein (cocktail) to lipid ratios. Effects of these parameters on nanosomes physical characteristics and stability in biological fluids are determined by constructing CNAP in nanosomes of different sizes ranging from 100 nm to 200 nm using various lipid materials (phosphatidylcholine and cholesterol), suspended and sterilized through 0.22 μm filters. Nanosomes size distribution, mean size, and standard deviation are analyzed by a submicron particle analyzer (Coulter Electronics, Inc., Model N4MD). This compute-controlled, multiple-angled instrument utilizes a laser beam to sense light scattering by particles. Latex spheres and empty nanosomes, prepared with only the lipids but not the therapeutics, are used as controls for these studies. Therapeutic content analysis is done through recovery experiments by analytical methods as HPLC, ELSD and LC/MS/MS.

CNAP delivers combination therapeutics that induce resistance to HIV infection in CD4+ cells by knocking out CCR5 coreceptor expression. In addition, the nanosomes contain the natural CCR5 ligand, CCL5, that is known to block HIV entry to target cells by specifically binding to CCR5. Comprehensive targeting is completed in a HIV latency model in which mice are treated with CNAP coated antibodies (anti-PDL-1, CD44, or CD54) to target resting CD4 T-cells or to stimulate immune cells non-specifically.

In Vitro Cytotoxicity And Efficacy Studies: The functional utility of the CNAP platform depends on delivering a therapeutic cocktail that is tissue specific for any given disease without toxic or off-target effects. Thus, cytotoxicity of the CNAP both in tissue culture and in a mice model of HIV latency are important. Cytotoxicity and growth inhibition in static and growing cell cultures are determined using the methyl tetrazolium (MTT) reduction assays. These tests are performed by treatment of the respective cells with various concentrations of CNAP. Control tests comprise treatment with ‘mock’ drugs or empty nanosomes. Toxicity data are collected over a 72-hour period. Growth inhibition is derived as a % value on this basis: [1−(A/B)]×100, where A=absorbance 570 nm of treated cells, and B=absorbance at 570 nm of control cells. The data are be used to calculate IC₅₀'s. The in vitro studies for CCR5 nanosomes are completed in PBMCs as the most relevant types of cells considering the biology of HIV and the proposed route of administration of the nanoparticles i.e. i.v. Efficacy are determined by monitoring the expression of the delta CCR5 molecule on the cell surface by FACS analysis followed by western blotting, if necessary. Subsequently, the resistance of the cells with altered expression of CCR5 are determined by infecting them with CCR5-tropic HIV-1 strains such as BaL and/or ADA and measuring virus replication by p24 antigen assay and/or qPCR for viral RNA. Model cell lines that express only CCR5 are also used.

In Vivo Toxicity. Biodistribution in Mice: Limited data suggest a lymphatic tissue distribution of liposomes at just over 75% after s.c. injection, although liposome co-encapsulation potentially enhanced distribution and potency in anticancer therapy. NOD.Cg-PrkcdscidlL2rgtmlWij/SzJ (NOD-SCID IL2rg−/−, or HSC-NSG) mice are used for validation in immunologically nude humanized environment. Adult Tg(UBC-CCR5,-CD4)19Mnz mice (n=40) expressing human CD4 and CCR5 under a luciferase reporter (The Jackson Laboratory) are treated with CNAP (10, 20,50 and 100 nM or vehicle control; n=4 per dose), via i.v. injections, for 25 weeks and monitored daily for morbidity and mortality. An equal set of control mice are treated with non-encapsulated drug components of the CNAP for comparison. Blood is obtained from the retro-orbital vein weekly until week 25. On week 25, animals are anesthetized with ketamine-xylazine and blood collected by cardiac puncture. Blood chemistry profiles (Synchron CX5CE chemical analyzer; Beckman) and blood counts (HESKA Vet ABC-Diff hematology analyzer) are measured. At necropsy, lymph nodes, liver, kidney, pancreas and bone marrow are collected for histopathological examination and biodistribution analysis by LC/MS/MS. The two highest nontoxic concentrations of CNAP are used in HSC-NSG) mice (n=24), which will be generated and reconstituted with human cells and in another set of Tg(UBC-CCR5,-CD4)19Mnz mice that will be used for gene regulation and infection studies. Successfully transplanted HSC-NSG mice (>5% of human CD45+ cells in peripheral blood) are treated daily with CNAP for 25 weeks and undergo same analyses as Tg(UBC-CCR5,-CD4)19Mnz. Endpoint analyses in HSC-NSG mice include measurements of human cell subsets (B cells, T cells and monocytes).

Infection and Gene Regulation Studies In Mice. Transgenic mice are highly susceptible to HIV infection, and are suitable for the therapeutic efficacy studies. Humanized mice are infected with a CCR5-specific strain of HIV-1 such as BaL or ADA and then followed up with ART until the infection becomes latent. Latency is confirmed by sacrificing a subset of mice and quantification of proviral DNA. Then mice are divided into the following treatment groups while continuing ART: (a) Control (no treatment) (b) CCR5 construct—4 doses. ‘Cure’ from latent HIV is determined by cessation of ART at the end of the treatment schedule and follow up for viral rebound. Plasma HIV RNA is monitored by RT-PCR, and CD4 and CCR5 expression is monitored by Flow Cytometry and p24 antigen by ELISA and Western Blot. Gene editing efficiency of the CRISPR/Cas9 constructs is assessed from cellular and proviral DNA by primer-specific sequencing.

Data Analyses: Data are analyzed using appropriate statistical approaches for specific set of variables. For example, a two-way analysis of variance (ANOVA) with Bonferroni's post-test correction is applied to the analyses of various scale variables using SPSS. Genetic sequence data is analyzed using appropriate bioinformatics tools.

Nucleic acids (NA) present a broad and novel potential for delivery of therapeutics to multiple disease sites and targets, making them versatile for the treatment of several conditions including (i) rare monogenetic disorders like Tay-Sach's syndrome; (ii) the rare familial Alzheimer's disease type 4; (iii) global infectious disease epidemics like the chronic human immunodeficiency virus (HIV) and, (iv) non communicable diseases like diabetes and cancer. While NAs carry this huge therapeutic potential, delivery to affected cells and tissues remains a major limiting factor.

In embodiments of the present invention, SuperFluids™ (SFS) technology is used for the manufacture and delivery of Critical Fluid Nanosomes (CFN™) in the particle size range of 100 nm to 200 nm. The CFN™ process harnesses the widely available environmentally safe gases as replacements for toxic organic solvents to manufacture highly pure nanoparticles. This process has been used successfully for efficient encapsulation of siRNA to target CD4 and CCR5 expression in cells. Further embodiments of this CFN™ technology platform are used to co-encapsulate combination NA co-loaded with guide RNA molecules in the aqueous core and targeting ligands on the surface of long circulating pegylated nanosomes (small, uniform liposomes).

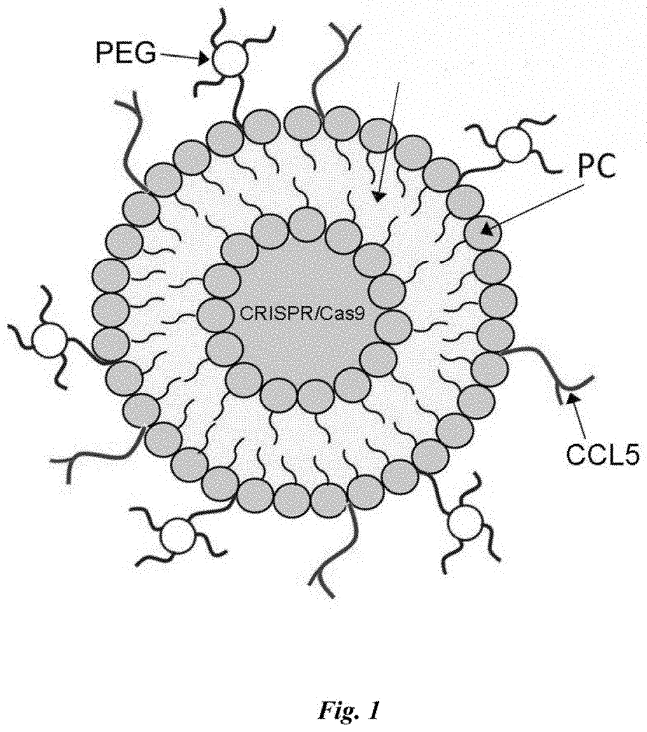

For latent HIV-1 infectious diseases, CFN™ nanosomes are used to encapsulate the clustered regularly interspaced short palindromic repeats (CRISPR) guide RNA pre-loaded on Cas9 protein to specifically knock down the HIV coreceptor CCR5 (CRISPR/Cas9-CCR5) expression and target them to CCR5 expressing CD4+ cells by coating the nanoparticles with CCL5, the natural ligand for CCR5. CRISPR/Cas9-CCR5 is designed to edit the CCR5 gene and introduce a 32 base pair deletion that occurs naturally in HIV resistant populations, and is encapsulated in the aqueous core of the nanoparticles. Specific delivery to CD4+ cells expressing CCR5 protein is made by the presence of CCL5 or alternative ligands mentioned before on the surface of the nanosomes. CCL5, aka RANTES protein is a CCR5 chemokine with a molecular weight of 9,900 that competes with HIV gp120 to bind CCR5. Coating CCL5 on nanoparticles is achieved by incorporating phosphatidylethanolamine into the lipid bilayer during synthesis of the nanoparticles. The ethanolamine on the surface of the nanoparticles is then cross-linked to the lysine residues in the RANTES protein by glutaraldehyde or other amine cross-linking chemistries. The Cas9 protein carries a Nuclear Localization Signal (NLS) to enable transport of the RNA-enzyme complex into the nucleus of the cells. The Cas9 protein has an affinity for and binds to gRNA sequences. After the complex is in the nucleus, the RNA component of CRISPR is in itself a robust targeting molecule, which leads Cas9 to the cognate DNA on the host genome. Hence, each of the components helps the active complex reach the target site of action. By deleting the CCR5 sequences in the target cells, this construct creates a population of CD4+ cells resistant to HIV-1 infection. Treatment of latently infected HIV-1 patients on ART with this formulation followed by treatment of the patient with LRAs with continuing ART is expected to provide a complete cure by elimination of activatable latently infected cells. Any infectious virus produced during latency reactivation would not be able to carry out multiple rounds of active infections and establish new latent infections since a proportion of the CD4+ cells would be resistant to HIV-1 infections due to the lack of expression of CCR5. Pegylation of the nanoparticles is introduced to increase residence and circulation time, and optimize bioavailability. This strategy carries a powerful application in gene and cell therapy for functional HIV-1 cure. Because the invention focuses on HIV-1 co-receptor, the platform targets cells expressing CCR5, mainly CD4+ T-cells, macrophages, monocytes and dendritic cells. This pegylated CFN™ construct (CNAP™), shown in FIG. 1, is versatile.

This invention draws from robust internal expertise and experience in the formulation and manufacturing of CFN™ nanoparticles. Even though this application is directed towards an HIV-1 cure, this NA drug delivery platform for CRISPR Cas9 is readily transferable to other diseases.

Thus, the present invention has been described in detail for an apparatus and methods for improved delivery of therapeutics and biologics for the treatment of diseases including latent HIV-1 viral disease with the understanding that those skilled in the art can modify and alter the detailed description herein without departing from the teaching. Therefore, the present invention should not be limited to the description but should encompass the subject matter of the claims that follow and their equivalents.

1. A method for delivering nuclear acid (NA) therapeutics and biologics to targeted cells for the treatment of diseases, comprising: forming nanoparticles in the range of 100 nm to 200 nm, nanoparticles having an aqueous core and pegylated targeting ligands on the surface; co-encapsulating a nucleic acid (NA) and guide RNA in the aqueous core for treating a specific disease; and coating the surface of the nanoparticles with targeting ligands for a specific cell.

2. The method of claim 1, wherein the nuclear acid in the aqueous nanosomes core is CRISPR Cas9.

3. The method of claim 1, wherein NA-protein hybrids CCR5-CRISPR/Cas9 are in the aqueous core, and CCL5, a ligand for CCR5 receptor, is on the surface of the nanoparticles.

4. The method of claim 1 wherein NA-protein hybrids CCR5-CRISPR/Cas9 are in the aqueous core, and an alternate ligand for CCR5 receptor, such as CCL3, CCL4, CCL8, truncated CCL5, modified CCL5 or small molecule inhibitors of HIV gp120 binding to CCR5 such as Maraviroc, is on the surface of the nanoparticles.

5. The method of claim 1, wherein NA-protein hybrids CCR5-CRISPR/Cas9 are in the aqueous core, and SDF1 (aka CXCL12), the ligand for CXCR4 receptor, is on the surface of the nanoparticles.

6. The method of claim 1 wherein NA-protein hybrids CCR5-CRISPR/Cas9 are in the aqueous core, and an alternate ligand for CXCR4 receptor, such as the different isoforms of CXCL12, 7 of which have been identified so far, or small molecule inhibitors of CXCL12 binding to CXCR4 such as Plerixafor, is on the surface of the nanoparticles.

7. The method of claim 1, wherein the nanoparticle is coated with polyethylene glycol (PEG) to increase residence or circulation time of the therapeutic in the body.

8. The method of claim 1, wherein the nucleic acid in the aqueous nanosome core is specific for the treatment of a disease selected from the group consisting of HIV-1, Alzheimer's disease, diabetes, and cancer.

9. The method of claim 7, wherein the ligand coating the surface of the nanoparticle is specific for the targeted cells of the selected disease consisting of HIV-1, Alzheimer's disease, diabetes, and cancer.

10. The method of claim 1, wherein the co-encapsulation includes a CFN combination nucleic acid (NA)-protein therapeutic that is PEGylated (CNAP), and targeted by cell and genome-specific RNA molecules in small nanosomes for delivering complex drug formulations.

11. An apparatus for making critical fluid nanoparticles for delivering encapsulated therapeutics and biologics to targeted disease cells, comprising
a. a circulation loop for forming the phospholipid solution with a supercritical, critical or near critical fluid;
b. a pressure vessel, in fluid communication with the circulation loop, for containing a mixture of an aqueous solution of nucleic acid and a phospholipid solution with a supercritical, critical or near critical fluid;
c. an injection nozzle in fluid communication with the pressure vessel for receiving the mixture and releasing the mixture as a stream into a decompression liquid;
d. a decompression vessel in fluid communication with the injection nozzle for holding a decompression liquid and receiving the mixture as a stream, wherein
e. the stream forms one or more nanoparticles in the decompression liquid, wherein the nanoparticles co-encapsulate nuclear acid with guide RNA molecules in the aqueous nanosome core; and
f. a coating bath for coating the surface of the nanoparticles with ligands for targeting specific disease cells.

12. The apparatus of claim 11, wherein the particle size of the nanoparticles is in the range of 100 nm to 200 nm.

13. The apparatus of claim 11, wherein the therapeutics and biologics are for treating HIV.

14. The apparatus of claim 11 wherein the nucleic acid and guide RNA are CRISPR/Cas9.

15. The apparatus of claim 11, where the targeting ligands coating the surface of the nanoparticles are CCL5, the ligand for CCR5 receptor.

16. The apparatus of claim 11, where the targeting ligands coating the surface of the nanoparticles are other protein or small molecules that bind to CCR5, such as CCL3, CCL4, CCL8, truncated CCL5, modified CCL5 or Maraviroc.

17. The apparatus of claim 11, where the targeting ligands coating the surface of the nanoparticles are CXCL12, the ligand for CXCR4 receptor.

18. The apparatus of claim 11, where the targeting ligands coating the surface of the nanoparticles are protein or small molecules that bind to CXCR4 receptor, such as the different isoforms of CXCL12, 7 of which have been identified so far, or small molecule inhibitors of CXCL12 such as Plerixafor.

19. The apparatus of claim 11, wherein the nucleic acid (NA) in the aqueous nanosome core and the ligands on the nanosome surface are specific for the treatment of a disease selected from the group consisting of HIV-1, Alzheimer's disease, diabetes, and cancer.

20. The apparatus of claim 11, wherein the aqueous solution comprises multiple nucleic acids (NAs), proteins and small molecules, and wherein the formed critical fluid nanoparticles contain multiple therapeutics in the aqueous core and/or in the non-aqueous matrix in a single nanosomal therapeutic cocktail.
